(19) United States
(12) Patent Application Publication
(10) Pub. No.: US 2010/0151097 A1
(43) Pub. Date: Jun. 17, 2010

(54) OPTIMIZATION OF COLICIN PRODUCTION

(75) Inventor: Chad H. Stahl, Raleigh, NC (US)

Correspondence Address: BARNES & THORNBURG LLP, P.O. BOX 2786, CHICAGO, IL, 60690-2786, US

(73) Assignees: BIOTECHNOLOGY RESEARCH AND DEVELOPMENT CORPORATION, Peoria, IL (US); IOWA STATE UNIVERSITY RESEARCH FOUNDATION, INC., Ames, IA (US)

(21) Appl. No.: 12/598,626

(22) PCT Filed: Mar. 25, 2008
(86) PCT No.: PCT/US08/58121

§ 371 Date: Nov. 3, 2009

Related U.S. Application Data
(60) Provisional application No. 60/916,709, filed on May 8, 2007.

(51) Int. Cl. A23L 3/3463; C12N 1/21; C12N 15/74; C07K 1/22; C12P 21/06

(52) U.S. Cl. 426/331; 435/252.33; 435/488; 530/413; 435/69.1; 426/335

(57) ABSTRACT

High colicin producing bacteria strains are produced by introducing into a host cell multiple copies of a plasmid containing a colicin gene. A suitable host cell is a bacterium strain, Escherichia coli K-12 and examples of plasmids are pColE1-K53 or pColN-284.

Strains of Escherichia coli are described that produce high levels of colicin.

### BACKGROUND

Several diseases in swine are known to be caused by Escherichia coli infections, for example, post-weaning diarrhea and edema disease. These diseases can cause substantial losses to the producers due to both mortality and mobidity. The E. coli strains F4 (K88) and F18 are known to be the primary strains causing these diseases. Attempts to prevent the spread of the diseases rely on the use of prophylactic antibiotic treatments. However, the traditional prophylactic antibiotic treatments are not effective on certain strains of disease-causing E. coli due to the resistance developed in these strains.

An alternative to the traditional antibiotic treatment is the use of bacteriocins, which are antibiotic proteins produced by certain strains of Entero-bacteriaceae and are known to be active against the same or closely related members of the same family of bacteria. Colicins are divided into two groups according to their cross-resistance patterns. Group A: A, E1, E2, E3, K, L, N, S4, and X and Group B: B, D, g, H, I, Ia, M, Q, S, and V.

Colicins E1 and N have been shown to be effective in inhibiting the growth of the disease causing E. coli strains F4 (K88) and F18.

### SUMMARY

Colicin producing cultures of bacterium were capable of producing high levels of colicin. The cultures included Escherichia coli host cells transformed with multiple copies of a plasmid containing a colicin gene. The transformed cells produced about 30 to about 50 fold higher levels of colicin than the original host cells. Suitable host cells include Escherichia coli-K12, containing pColE1-K53 or pColN-284 plasmids, for example, or any other plasmid that includes a colicin gene and appropriate expression regulatory elements, and preferably, also a gene conferring resistance to colicin. Plasmids used in the transformation include pColE1-K53 or pColN-284, which may be extracted from the host cells. The colcicin produced by the cultures of the present invention include colicin E1 or colicin N, but other colicins may also be desirable.

In one embodiment of the invention, an Escherichia coli strain is K12 subCHSE1 or an Escherichia coli strain K12 CHSN.

In another aspect of the invention, a method for producing a high colicin producing culture, includes:

(a) introducing multiple copies of a plasmid containing a colicin gene into suitable host cells to produce transformed cells;

(b) subjecting the transformed cells to selection media containing increasing concentrations of colicin; and

(c) selecting transformed cells that grow on media containing more than 1 mg/L colicin, and producing more than 30 fold higher colicin than the host cells.

A suitable host cell is Escherichia coli-K12.

A method for purification of colicin includes:

(a) growing the culture of E. coli strains, for example, K12sub CHSE1 and K12CHSN, in a liquid medium to reach an OD₆₀₀ of about 0.85 to about 0.95;

(b) adding 0.2 μg/L of mitomycin C to the growing culture;

(c) obtaining a supernatant from step (b);

(d) passing the supernatant through a DEAE cellulose column;

(e) concentrating supernatant from step (d) by ultracentrifugation;

(f) desalting the concentrated supernatant against 20 mM Tris, pH 8;

(g) applying the protein solution to a Q-sepharose column; and

(e) eluting the colicin with a NaCl gradient.

### DETAILED DESCRIPTION

Super strains of bacteria are capable of producing very high levels of colicin, particularly colicin E1 and colicin N. Colicins E1 and N can be used as alternative antibiotics for treating of bacteria causing diseases in animals such as post weaning diarrhea and edema disease in swine. Colicins are also useful for washing meat and produce to minimize bacterial contamination.

Colicin is a protein encoded by a colicin gene located on a plasmid (pCol). By increasing the copy number of the colicin gene in bacterial cells, the cells produced much higher levels of colicin than in the host cells with the plasmid. The present disclosure describes procedures for producing the super strains of bacteria having multiple copies of pCol plasmid, and for producing and purifying colicins from the super strains of the bacteria.

In order to increase the copy number of the colicin gene, the plasmid DNA representing multiple copies of pCol was introduced into host cells. The host cells may be any suitable E. coli strain that is void of pCol or contains one or more copies of pCol. The suitable resulting transformed cells contain more copies of pCol than the original host cells.

The plasmid DNA containing a colicin gene may be isolated from any suitable source depending on the desired type of colicin. For example, E. coli 284 contains the plasmid pColN-284, carrying a colicin gene (cna) that encodes colicin N. E. coli K534 contains the plasmid pColE1-K53, carrying a colicin gene (cea) gene encoding colicin E1. Typically multiple copies (15-25) of pColE1-K53 or pColN-284 are present in a corresponding E. coli cell. The plasmids pColN-284 and pColE1-K53 have previously been isolated and transformed into E. coli K12 to produce E. coli K12 pColN-284 and E. coli K12 pColE1-K53 strains, respectively. Therefore, pColN-284 or pColE1-K53 DNA may be isolated from a E. coli K12 pColN-284 or E. coli K12 pColE1-K53 strain.

Typically, on the pCol plasmid, there is also an immunity gene that confers resistance to the colicin that is encoded by the colicin gene on the same plasmid. For example, pColN-284 contains an immunity gene that confers resistance to colicin N, and likewise, pColE1-K53 contains an immunity gene that confers resistance to colicin E1. The level of resistance to colicin likely depends partly on the number of copies of the immunity gene present. Therefore, the cells are able to tolerate higher concentrations of colicin if they have higher numbers of the pCol plasmid.

The methods for isolating bacterial plasmid DNA are well known in the art. Commercial reagent kits containing all the necessary reagents for cell lysis and plasmid DNA purification are commercially available.

The purified plasmid DNA may be introduced into the host cells using any suitable methods that are also well known in the art.

There are a number of methods known in the art for introducing the plasmid DNA into bacterial cells. For example, the transformation method may be performed using an electroporation technique that creates pores along the cell membrane allowing the plasmid DNA to enter the cells. Cells are then selected based on the resistance to a specific colicin. For example, cells that are transformed with pColN-284 are selected using media containing varying levels of colicin N, whereas cells transformed with pColE1 are selected using media containing varying levels of colicin E1. The strains that grow on in the media containing the highest level of colicin are selected. The selected cells are expected to contain multiple copies of pCol plasmid, exceeding the original copy number of the plasmid in the original host strain.

In an embodiment, a method for producing a high colicin producing strain includes the following steps:

(a) introducing multiple copies of a plasmid containing a colicin gene into Escherichia coli-K12 host cells to produce transformed cells;

(b) subjecting the transformed cells to selection media containing increasing concentrations of colicin; and

(c) selecting transformed cells that grow on media containing more than 100 mg/L colicin, and producing more than 30 fold higher colicin than the host cells.

In a more specific embodiment, the host cell is Escherichia coli-K12 pColN-284 and the plasmid is pColN-284 isolated from E. coli K12 pColN-284. In an alternative embodiment, the host cell is E. coli K12 pColE1-K53, and the plasmid is pColE1-K53 isolated from E. coli K12 pColE1-K53. Both strains of host cells may be obtained from the National Collection of Type Cultures (Public Health Laboratory Service, London, England).

Each selected strain of E. coli transformed with pColN-284 is expected to contain a high number (more than about 15 to 25) of pColN-284 and shows resistance to a high concentration (>100 mg/L) of colicin N. Similarly, each selected strain of E. coli transformed with pColN-284 is expected to contain a high number (more than about 15 to 25) copies of pColE1-K53 and shows resistance to a high concentration (>100 mg/L) of colicin E1. The selected strains are capable of producing high levels of colicin which may reach about 30 to about 50 fold higher than the host cells.

One selected strain resulting from the transformation of Escherichia coli-K12 pColN-284 with the plasmid pColN-284 is designated Escherichia coli strain K-12 CHSN.

Another selected strain resulting from the transformation of Escherichia coli-K12 pColE1-K53 with the plasmid pColE1-K53 is designated Escherichia coli strain K-12CHSE1.

For colicin production, a selected strain of E. coli described herein is grown in a LB medium under a standard condition (37° C., with shaking). The cells are grown to an OD₆₀₀ of about 0.85 to about 0.95 before a determined amount of mitomycin C is added to the cells. The cells are separated from the supernatant by centrifigation then colicin is extracted and purified from the cell-free supernatant. It is also possible to use any method suitable for protein extraction and purification.

### Example 1

The parent bacteria strains: Two individual E. coli strains E. coli K12, one containing the plasmid pColE1 (herein after referred to as E. coli K12-pColE1) and the other containing the plasmid pColN-284 (herein after referred to as E. coli K12-pCoIN-284) were obtained from the National Collection of Type Cultures (Public Health Laboratory Service, London, England). Each bacteria culture was grown in a Luria Broth (LB) at 37° C. with shaking.

### Example 2

Preparation of the pColE1-K53 or pColN-284 plasmid: The plasmid pColE1 DNA was isolated from E. coli K12-pColE1-K53 culture and the plasmid pCoIN-284 DNA was isolated from E. coli K12-pCoIN-284 culture using an alkaline lysis method known in the art.

### Example 3

Production of Transformed Strain: The isolated plasmid pColE1 DNA was introduced into E. coli K12-pColE1-K53 and the isolated plasmid pCoIN-284 DNA was introduced into E. coli K12-pCoIN-284 using a standard electroporation procedure with the settings of 2 kV, 25 mF, and 200 ohms. The electroporated cells were allowed to recover in LB medium for one hour and were then diluted 1:100 into LB medium containing various does (0.1, 0.25, 0.5, 0.75, and 1 mg/mL) of the corresponding type of colicin and were incubated at 37° C. with shaking overnight. The highest colicin concentration media that demonstrated growth was then diluted 1:100 into a gradient of higher concentrations of the corresponding colicin in LB medium and allowed to grow overnight. After four days, colicin resistant colonies were streaked on to an LB agar medium. Individual colonies were picked and inoculated into LB broth and split into two cultures. One culture was used to test for colicin production and the other was used to prepare a frozen stock culture.

### Example 4

Colicin Production and Purification: The selected cultures were inoculated into the LB broth to a starting optical density at 600 nm (OD₆₀₀) of about 0.1 and incubated the cultures with shaking at 37° C. Colicin production was induced when the cultures reached an OD₆₀₀ of 0.85-0.95 by the addition of 0.2 microgram of mitomycin C (Sigma Scientific) per milliliter of culture. The cultures were allowed to remain in the shaking incubator for 4.5-5.5 hours. The cell-free supernatant was obtained by centrifugation and passed through a DEAE cellulose column and then concentrated by ultracentrifugation through a 10 kD molecular weight cut-off membrane. The concentrate was then exhaustively desalted against 20 mM Tris, pH 8 and then applied to a Q-sepharose column. The colicin was eluted with a NaCl gradient by using AKTAprime chromatography system (Amersham Bioscience), and fractions containing the highest concentration of colicin were pooled and concentrated and desalted by ultrafiltration. The protein concentrations of the pooled samples were determined (Lowry method), known to those of skill in the art.

### Example 5

Determination of percentage of colicin: The percentage of colicin was determined using the procedure described in Stahl et al. 2004. Briefly, the colicin concentration was determined by densitometry after sodium dodecyl sulfate-polyacrylamide gel electrophoresis followed by Coomassie blue staining with a 16 bit megapixel charge-coupled device camera, Fluor Chem 8800, and Fluor Chem IS800 software (Alpha Innotech, San Leandro, Calif.). Yields of 1.1 mg of purified ColN/liter of culture and 7.6 mg of purified ColE1.liter of culture were obtained from the parent strains. The purity of the ColN and ColE1 isolates were 30 and 85%, respectively. The yields of the selected strains were about 33 to 50 fold of the parent strains. (250 mg/L for colE1 and 55 mg/L for colN). The culture that was selected from the parent E. coli K12-pColE1-K53 was named E. coli K12 CHSE1, whereas the culture that was selected from the E. coli K12-pCoIN-284 parent strain was named E. coli K12 CHSN.

While the invention has been illustrated and described in detail the same is to be considered as illustrative and not restrictive in character. It should be understood that only the exemplary embodiments have been shown and described and that all changes and modifications that come within the spirit of the invention are desired to be protected.

### PUBLICATIONS

The publications listed are incorporated by reference to the extent the relate materials and/or methods described herein.
Stahl et al. (2004) “Inhibitory Activities of Colicins against Escherichia coli Strains Responsible for Postweaning Diarrhea and Edema Disease,” Antimicrobial. Agents And Chemoterapy, p. 3119-3121.
Schwartz, Stanley A. and Helsinke, Donald R. (1971) “Purification and Characterization of Colicin E₁,” J. Biol. Chem. 246 (20): 6318-6327 US2006/0154338 A1.

1. A colicin producing bacterium strain comprising:
Escherichia coli host cells transformed with multiple copies of a plasmid containing a colicin gene, the transformed cells capable of producing about 30 to about 50 fold higher levels of colicin than native colicin E1 producing E. coli.

2. The strain of claim 1, wherein the host cells are Escherichia coli-K12.

3. The strain of claim 1, wherein the host cells are Escherichia coli-K12 containing pColE1-K53 or pColN-284.

4. The strain of claim 1, wherein the plasmid containing a colicin gene is pColE1-K53.

5. The strain of claim 1, wherein the plasmid containing a colicin gene is pColN-284.

6. The strain of claim 1, wherein the colicin gene encodes colicin E1 or colicin N.

7. An Escherichia coli strain designated K-12 CHSE1 that produces higher levels of colicin than do native colicin E1 producing E. coli, when comparisons are made between induced cells.

8. An Escherichia coli strain K-12CHSN that produces higher level of colicin than do native colicin E1 producing E. coli, when comparisons are made between induced cells.

9. A method for producing a high colicin producing strain, comprising:
(a) introducing multiple copies of a plasmid containing a colicin gene into Escherichia coli-K12 host cells to produce transformed cells;
(b) subjecting the transformed cells to selection media containing increasing concentrations of colicin; and
(c) selecting transformed cells that grow on media containing more than 100 mg/L colicin, and producing more than 30 fold higher colicin than the host cells.

10. A method for purification of colicin comprising:
(a) culturing the strain of claim 7 in a liquid medium to reach an OD₆₀₀ of about 0.85 to about 0.95;
(b) adding 0.2 μg/L of mitomycin C to the growing culture in step (a);
(c) obtaining a supernatant from step (b);
(d) passing the supernatant through a DEAE cellulose column;
(e) concentrating supernatant from step (d) by ultracentrifugation;
(f) desalting the concentrated supernatant against 20 mM Tris, pH 8;
(g) applying the protein solution to a Q-sepharose column; and
(e) eluting the colicin with a NaCl gradient.

11. (canceled)

12. A method for purification of colicin comprising:
(a) culturing the strain of claim 8 in a liquid medium to reach an OD₆₀₀ of about 0.85 to about 0.95;
(b) adding 0.2 μg/L of mitomycin C to the growing culture in step (a);
(c) obtaining a supernatant from step (b);
(d) passing the supernatant through a DEAE cellulose column;
(e) concentrating supernatant from step (d) by ultracentrifugation;
(f) desalting the concentrated supernatant against 20 mM Tris, pH 8;
(g) applying the protein solution to a Q-sepharose column; and
(h) eluting the colicin with a NaCl gradient.

13. A method to minimize bacterial contamination of meat, produce, or other RTE products, the method comprising:
(a) obtaining purified colicin from a strain of claim 1;
(b) washing the meat, produce or other RTE products to minimize bacterial contamination.

14. A method to minimize bacterial contamination of meat, produce, or other RTE products, the method comprising:
(a) obtaining purified colicin from a strain of claim 7;
(b) washing the meat, produce or other RTE products to minimize bacterial contamination.

15. A method to minimize bacterial contamination of meat, produce, or other RTE products, the method comprising:
(a) obtaining purified colicin from a strain of claim 8;
(b) washing the meat, produce or other RTE products to minimize bacterial contamination.
